(19) United States
(12) Patent Application Publication
(10) Pub. No.: US 2020/0113798 A1
(43) Pub. Date: Apr. 16, 2020

(54) TRANSPARENT COSMETIC COMPOSITION HAVING ULTRAVIOLET SCREENING FUNCTION

(71) Applicant: KOLMAR KOREA CO., LTD., Sejong-si (KR)

(72) Inventor: Jin Young KIM, Seoul (KR)

(21) Appl. No.: 16/626,784

(22) PCT Filed: May 18, 2018
(86) PCT No.: PCT/KR2018/005706

§ 371 Date: Dec. 26, 2019

(30) Foreign Application Priority Data
Jun. 29, 2017 (KR) 10-2017-0082677

(51) Int. Cl. A61K 8/37; A61K 8/89; A61K 8/49; A61K 8/891; A61K 8/41; A61Q 17/04; A61Q 19/02

(52) U.S. Cl. CPC A61K 8/37; A61K 8/89; A61K 8/4966; A61Q 19/02; A61K 8/415; A61Q 17/04; A61K 8/891

(57) ABSTRACT

A transparent cosmetic composition has an ultraviolet-screening function. The transparent cosmetic composition includes an elastomer, an oil and an ultraviolet-screening agent. The transparent cosmetic composition has a transparent formulation, forms no white cast, and has a high ultraviolet-screening effect. The transparent cosmetic composition can also function as a makeup primer.

### TECHNICAL FIELD

The present invention relates to a transparent cosmetic composition having an ultraviolet-screening function.

### BACKGROUND ART

Ultraviolet light is radiation in the wavelength range of 280 to 400 nm, and an appropriate amount of ultraviolet light enables actions like a skin disinfection effect, inflammation relief, vitamin D synthesis and the like, but when exposed to excessive ultraviolet light, aging of the skin, such as erythema formation, generation of blemishes, freckles and uneven pigmentation due to melanin overproduction, wrinkles, and skin sagging and looseness, may be promoted. Accordingly, the skin is protected by applying various types of ultraviolet-screening cosmetics onto the skin.

In general, ultraviolet-screening cosmetics include inorganic ultraviolet-screening agents, which prevent ultraviolet light from penetrating into the skin by scattering and reflecting ultraviolet light using inorganic materials such as titanium dioxide, zinc oxide, etc., and organic ultraviolet-screening agents, which prevent ultraviolet light from penetrating into the skin by the chemical absorption action using organic materials such as ethylhexyl methoxycinnamate, ethylhexyl salicylate, homosalate, octocrylene and the like.

In this regard, Korean Patent No. 10-1444086 discloses a cosmetic composition having a multilayer structure with an ultraviolet-screening function, including an aqueous layer composed of a surfactant and a salt, which are mixed together, and an ultraviolet-screening layer including titanium dioxide or octocrylene as an ultraviolet-screening agent, and Korean Patent Application Publication No. 10-2011-0012188 discloses an ultraviolet-screening cosmetic composition containing 2-methoxycinnamaldehyde as an active ingredient and octocrylene or titanium dioxide as an ultraviolet-screening agent.

Representative examples of inorganic ultraviolet-screening agents include titanium dioxide and zinc oxide, which are in the form of white granules, and thus, when included in ultraviolet-screening cosmetics, not only is the transparency of the cosmetics deteriorated, but there is also a problem of reducing the use sensation by generating a white cast. Furthermore, octocrylene causes allergies by irritating the skin, and also causes problems such as acne, inflammation, flushing, etc. Hence, the development of hypoallergenic ultraviolet-screening cosmetics, having a transparent formulation and causing almost no white cast, is required.

With the goal of solving the aforementioned problems, cream-, lotion-, spray- and gel-type ultraviolet-screening cosmetics using only organic ultraviolet-screening agents, such as phenylbenzimidazole sulfonic acid, ethylhexyl methoxycinnamate, ethylhexyl salicylate, homosalate, isoamyl p-methoxycinnamate, ethylhexyl triazone, butyl methoxydibenzoylmethane, bis-ethylhexyloxyphenol methoxyphenyl triazine, diethylamino hydroxybenzoyl hexyl benzoate, and the like, are being developed. However, in order to realize a high ultraviolet-screening effect, the use of excess organic ultraviolet-screening agent may cause a poor use sensation, such as a strongly greasy or sticky feeling, and does not reduce the white cast generated upon application onto the skin, and moreover, the resulting formulation is still opaque.

Therefore, it is necessary to develop a cosmetic that has a transparent formulation and a high ultraviolet-screening effect without causing a white cast. Furthermore, in recent years, there is increasing demand for multifunctional cosmetics that implement multiple functions at the same time, and thus, the development of cosmetics that may function as a makeup primer that improves the coverage of skin pores and irregularities and adhesion to the skin, as well as ultraviolet-screening performance, is also required.

### DISCLOSURE

### Technical Problem

Accordingly, an objective of the present invention is to provide a transparent cosmetic composition, which has a transparent formulation and a high ultraviolet-screening effect without generating a white cast, and which may also function as a makeup primer.

The objectives of the present invention are not limited to the foregoing, and other objectives will be able to be derived from the following description.

### Technical Solution

In order to accomplish the above objective, the present invention provides a transparent cosmetic composition including an elastomer, an oil, and an organic ultraviolet-screening agent composition.

The transparent cosmetic composition may include, based on the total weight of the transparent cosmetic composition, 35 to 70 wt % of the elastomer, 1 to 30 wt % of the oil and 5 to 40 wt % of the organic ultraviolet-screening agent composition.

The elastomer may include at least one selected from the group consisting of (A) a dimethicone/bis-isobutyl PPG-20 crosspolymer and (B) a PEG-12 dimethicone/PPG-20 crosspolymer.

The organic ultraviolet-screening agent composition may include at least one selected from the group consisting of ethylhexyl methoxycinnamate, ethylhexyl salicylate, homosalate, polysilicone-15, isoamyl p-methoxycinnamate, ethylhexyl triazone, butyl methoxydibenzoylmethane, bis-ethylhexyloxyphenol methoxyphenyl triazine and diethylamino hydroxybenzoyl hexyl benzoate.

The organic ultraviolet-screening agent composition may include, based on the total weight of the transparent cosmetic composition, 3 to 20 wt % of ethylhexyl methoxycinnamate, 3 to 20 wt % of ethylhexyl salicylate, 0.1 to 20 wt % of homosalate, 0.1 to 20 wt % of polysilicone-15, 0.1 to wt % of isoamyl p-methoxycinnamate, 0.1 to 10 wt % of ethylhexyl triazone, 0.1 to 10 wt % of butyl methoxydibenzoylmethane, 0.1 to 10 wt % of bis-ethylhexyloxyphenol methoxyphenyl triazine, and 0.1 to 10 wt % of diethylamino hydroxybenzoyl hexyl benzoate.

The oil may include at least one selected from the group consisting of butyloctyl salicylate, dibutyl adipate, butylene glycol dicaprylate/dicaprate, caprylyl methicone, isododecane, isohexadecane, C15-19 alkane and dicaprylyl carbonate.

The transparent cosmetic composition may further include at least one additive selected from the group consisting of a preservative, a whitening agent, an antioxidant, a skin-conditioning agent, and a surfactant.

### Advantageous Effects

According to the present invention, a transparent cosmetic composition has a transparent formulation and has a high ultraviolet-screening effect without generating a white cast.

In particular, the transparent cosmetic composition of the present invention has a transparent formulation, causes no white cast and can exhibit a high ultraviolet-screening effect, despite the use of at least one organic ultraviolet-screening agent selected from the group consisting of ethylhexyl methoxycinnamate, ethylhexyl salicylate, homosalate, polysilicone-15, isoamyl p-methoxycinnamate, ethylhexyl triazone, butyl methoxydibenzoylmethane, bis-ethylhexyloxyphenol methoxyphenyl triazine and diethylamino hydroxybenzoyl hexyl benzoate.

Moreover, the transparent cosmetic composition of the present invention can exhibit superior coverage of irregularities and pores of the skin and high skin adhesion, and can thus function both as an ultraviolet-screening agent and a makeup primer.

### BRIEF DESCRIPTION OF THE DRAWINGS

FIG. 1 shows Example 1;

FIG. 2 shows Example 2;

FIG. 3 shows Example 3 (left) and Example 4 (right);

FIG. 4 shows Comparative Example 1;

FIG. 5 shows Comparative Example 2;

FIG. 6 shows Comparative Example 3;

FIG. 7 shows Comparative Example 4;

FIG. 8 shows Comparative Example 5;

FIG. 9 shows Comparative Example 6;

FIG. 10 shows Comparative Example 7;

FIG. 11 shows Comparative Example 9;

FIG. 12 shows Comparative Example 10;

FIG. 13 shows Comparative Example 12;

FIG. 14 shows Comparative Example 12;

FIG. 15 shows Comparative Example 13;

FIG. 16 shows Comparative Example 14; and

FIG. 17 shows Comparative Example 15 (left), Comparative Example 16, Comparative Example 17, and Comparative Example 18 (right).

### BEST MODE

The present invention will be described more fully hereinafter with reference to the accompanying drawings, in which embodiments of the present invention are shown so as to be easily understood by those skilled in the art. However, the present invention may be embodied in many different forms, but is not limited to the embodiments described herein. In order to clearly illustrate the present invention, parts not related to the description are omitted in the drawings.

It is to be understood that the terms or words used in the specification and claims of the present invention are not to be construed in a conventional or dictionary sense, and that the inventors can properly define the concept of a term to describe their invention in the best possible way. Accordingly, the present invention should be construed as adhering to meanings and concepts consistent with the technical idea of the present invention.

In the specification of the present invention, when any portion “includes” any component, this means that the portion does not exclude other components, but may further include other components unless otherwise stated.

In the specification of the present invention, “A and/or B” means either A or B or A and B.

In the specification of the present invention, a “transparent cosmetic composition having an ultraviolet-screening function” may simply be referred to as a “transparent cosmetic composition”.

Hereinafter, the present invention is specifically described with reference to the accompanying drawings, but the present invention is not limited thereto.

The present invention pertains to a transparent cosmetic composition having an ultraviolet-screening function.

According to an embodiment of the present invention, the transparent cosmetic composition includes an elastomer, an oil and an organic ultraviolet-screening agent composition, and thus has a transparent formulation and a high ultraviolet-screening effect without causing a white cast.

In particular, the transparent cosmetic composition according to the present embodiment includes, based on the total weight of the transparent cosmetic composition, 35 to 70 wt % of the elastomer, 1 to 30 wt % of the oil and 5 to 40 wt % of the organic ultraviolet-screening agent composition, and may thus have a transparent formulation, no white cast and a high ultraviolet-screening effect, and may also function as a makeup primer. Moreover, the transparent cosmetic composition according to the present embodiment may have a smooth and refreshing feeling but low aggregation, which may facilitate makeup and additional application, and may be excellent in helping makeup last a long time.

The elastomer is a kind of polymer, and the organic ultraviolet-screening agent composition may be stably dispersed and dissolved in the elastomer of the present embodiment. In the present embodiment, the elastomer may include organic silicone, particularly at least one selected from the group consisting of (A) a dimethicone/bis-isobutyl PPG-20 crosspolymer and (B) a PEG-12 dimethicone/PPG-20 crosspolymer.

In particular, the elastomer including at least one selected from the group consisting of (A) and (B) as above may exhibit high compatibility and stability such that the organic ultraviolet-screening agent composition may be sufficiently dispersed or dissolved in the elastomer, and thus the organic ultraviolet-screening agent composition may not precipitate in the form of granules. Accordingly, the transparent cosmetic composition of the present embodiment may have a transparent formulation and may not generate a white cast. Furthermore, it is responsible for ultraviolet-screening performance and may function as a makeup primer.

The transparent cosmetic composition of the present embodiment may include the elastomer in an amount of 35 to 70 wt %, preferably 38 to 65 wt %, and more preferably 40 to 60 wt %, based on the total weight of the transparent cosmetic composition. If the amount of the elastomer is less than 35 wt % based on the total weight of the transparent cosmetic composition of the present embodiment, the transparency of cosmetics may decrease and the resulting formulation is unstable, thus causing layer separation, thereby deteriorating ultraviolet-screening and makeup primer functions. On the other hand, if the amount of the elastomer exceeds 70 wt % based on the total weight of the transparent cosmetic composition of the present embodiment, the organic ultraviolet-screening agent composition may precipitate, and the use sensation, transparency and ultraviolet-screening effect may deteriorate.

The organic ultraviolet-screening agent composition may include at least one selected from the group consisting of ethylhexyl methoxycinnamate, ethylhexyl salicylate, homosalate, polysilicone-15, isoamyl p-methoxycinnamate, ethylhexyl triazone, butyl methoxydibenzoylmethane, bis-ethylhexyloxyphenol methoxyphenyl triazine and diethylamino hydroxybenzoyl hexyl benzoate.

In particular, the organic ultraviolet-screening agent composition of the present embodiment may include, based on the total weight of the transparent cosmetic composition, 3 to 20 wt % of ethylhexyl methoxycinnamate, 3 to 20 wt % of ethylhexyl salicylate, 0.1 to 20 wt % of homosalate, 0.1 to 20 wt % of polysilicone-15, 0.1 to 20 wt % of isoamyl p-methoxycinnamate, 0.1 to 10 wt % of ethylhexyl triazone, 0.1 to 10 wt % of butyl methoxydibenzoylmethane, 0.1 to 10 wt % of bis-ethylhexyloxyphenol methoxyphenyl triazine, and 0.1 to 10 wt % of diethylamino hydroxybenzoyl hexyl benzoate.

The transparent cosmetic composition of the present embodiment may include the organic ultraviolet-screening agent composition, the components of which are mixed at the above ratio, whereby the organic ultraviolet-screening agent components in solid phase are sufficiently dissolved and thus transparency and a high ultraviolet-screening effect may be exhibited without causing a white cast.

In the organic ultraviolet-screening agent composition, ethylhexyl methoxycinnamate, ethylhexyl salicylate, homosalate, polysilicone-15, isoamyl p-methoxycinnamate and ethylhexyl triazone may function to block UVB, and butyl methoxydibenzoylmethane, bis-ethylhexyloxyphenol methoxyphenyl triazine and diethylamino hydroxybenzoyl hexyl benzoate may function to block UVA.

In the organic ultraviolet-screening agent composition, ethylhexyl triazone, butyl methoxydibenzoylmethane, bis-ethylhexyloxyphenol methoxyphenyl triazine and diethylamino hydroxybenzoyl hexyl benzoate are solid organic ultraviolet-screening agents. Also, in the organic ultraviolet-screening agent composition, ethylhexyl methoxycinnamate, ethylhexyl salicylate, homosalate, polysilicone-15 and isoamyl p-methoxycinnamate are liquid organic ultraviolet-screening agents.

In the organic ultraviolet-screening agent composition, when the mixing weight ratio of the total sum of the liquid organic ultraviolet-screening agent including at least one selected from the group consisting of ethylhexyl methoxycinnamate, ethylhexyl salicylate, homosalate, polysilicone-15 and isoamyl p-methoxycinnamate to the total sum of the solid organic ultraviolet-screening agent including at least one selected from the group consisting of ethylhexyl triazone, butyl methoxydibenzoylmethane, bis-ethylhexyloxyphenol methoxyphenyl triazine and diethylamino hydroxybenzoyl hexyl benzoate is 3.5-5:1, the solid organic ultraviolet-screening agent may be sufficiently dissolved and thus may not precipitate, and transparency may be further improved. Preferably, in the organic ultraviolet-screening agent composition, the mixing weight ratio of the liquid organic ultraviolet-screening agent to the solid organic ultraviolet-screening agent is 4:1.

If the mixing weight ratio of the liquid organic ultraviolet-screening agent to the solid organic ultraviolet-screening agent falls out of the above range and is less than the above lower limit, the organic ultraviolet-screening agent composition may precipitate, which is undesirable. On the other hand, if the mixing weight ratio of the liquid organic ultraviolet-screening agent to the solid organic ultraviolet-screening agent exceeds the above upper limit, the resulting cosmetic composition may become opaque or the use sensation thereof may deteriorate.

The transparent cosmetic composition of the present embodiment may include the organic ultraviolet-screening agent composition in an amount of 5 to 40 wt %, preferably 7 to 35 wt %, and more preferably 10 to 30 wt % based on the total weight of the transparent cosmetic composition. If the amount of the organic ultraviolet-screening agent composition is less than 5 wt % based on the total weight of the transparent cosmetic composition, the ultraviolet-screening effect may become insignificant. On the other hand, if the amount of the organic ultraviolet-screening agent composition exceeds 40 wt %, the organic ultraviolet-screening agent composition may precipitate in the form of granules, transparency may decrease, and moreover, a poor use sensation, such as greasiness or stickiness, may result.

The oil may aid the mixing of the elastomer and the organic ultraviolet-screening agent composition according to the present embodiment to thus improve the transparency of the transparent cosmetic composition of the present embodiment and the use sensation thereof. In the present embodiment, the oil may include at least one selected from the group consisting of butyloctyl salicylate, dibutyl adipate, butylene glycol dicaprylate/dicaprate, caprylyl methicone, isododecane, isohexadecane, C15-19 alkane and dicaprylyl carbonate.

The transparent cosmetic composition of the present embodiment may include the oil in an amount of 1 to 30 wt %, preferably 5 to 25 wt %, and more preferably 10 to 17 wt % based on the total weight of the transparent cosmetic composition. If the amount of the oil is less than 1 wt % based on the total weight of the transparent cosmetic composition, the elastomer and the organic ultraviolet-screening agent composition may not be sufficiently mixed, undesirably decreasing transparency or precipitating the organic ultraviolet-screening agent composition. On the other hand, if the amount of the oil exceeds 30 wt % based on the total weight of the transparent cosmetic composition, the use sensation, coverage and adhesion may deteriorate due to excessive oiliness, and moreover, the resulting formulation is unstable and layer separation may occur.

The transparent cosmetic composition of the present embodiment may further include a functional additive, a skin-conditioning agent, a preservative, a surfactant, etc. that have whitening, antioxidant, anti-wrinkling and moisturizing effects, in addition to the elastomer, the organic ultraviolet-screening agent composition and the oil, and the functional additive may be in the form of a powder, a liquid, a capsule, or the like.

The preservative may include, but is not limited to, phenoxyethanol, ethylhexylglycerin and the like, and may include all known preservatives applicable to cosmetic compositions.

The whitening component may include, but is not limited to, a licorice extract, Bisabolol and the like, and may include all known whitening components applicable to cosmetic compositions.

The antioxidant component and the skin-conditioning agent may include, but are not limited to, vitamin B5, tocopheryl acetate, hydroxyacetophenone and the like, and may include all known antioxidant components and skin-conditioning agents applicable to cosmetic compositions.

The surfactant may include, but is not limited to, cetyl PEG/PPG-10/1 dimethicone, lauryl PEG-9 polydimethylsiloxyethyl dimethicone and the like, and may include all known surfactants applicable to cosmetic compositions.

A better understanding of the transparent cosmetic composition having ultraviolet-screening performance according to the present invention will be given through the following examples, comparative examples and test examples. These examples are merely set forth to illustrate the present invention but are not to be construed as limiting the scope of the present invention.

### EXAMPLES

### Examples 1 to 6

Respective cosmetic compositions were prepared using components in the amounts shown in Table 1 below. In the following Table 1, Elastomer (A) is a dimethicone/bis-isobutyl PPG-20 crosspolymer and Elastomer (B) is a PEG-12 dimethicone/PPG-20 crosspolymer.

TABLE 1

| | Example (wt %) | | | | | |
|---|---|---|---|---|---|---|
| | 1 | 2 | 3 | 4 | 5 | 6 |
| Elastomer (A) | — | 55.0 | 25.0 | 30.0 | — | — |
| Elastomer (B) | 55.0 | — | 30.0 | 25.0 | 55.0 | 55.0 |
| Ethylhexyl methoxycinnamate | 5.0 | 5.0 | 5.0 | 5.0 | 5.0 | 7.0 |
| Ethylhexyl salicylate | 5.0 | 5.0 | 5.0 | 5.0 | 5.0 | 5.0 |
| Homosalate | 7.0 | 7.0 | 7.0 | 7.0 | 6.5 | 7.0 |
| Polysilicone-15 | 4.0 | 4.0 | 4.0 | 4.0 | 4.0 | 4.0 |
| Isoamyl p-methoxycinnamate | 3.0 | 3.0 | 3.0 | 3.0 | 3.0 | 2.0 |
| Ethylhexyl triazone | 1.5 | 1.5 | 1.5 | 1.5 | 1.0 | 1.5 |
| Butyl | 1.0 | 1.0 | 1.0 | 1.0 | 3.0 | — |
| methoxydibenzoylmethane | | | | | | |
| Bis-ethylhexyloxyphenol | 2.5 | 2.5 | 2.5 | 2.5 | 1.5 | 2.0 |
| methoxyphenyl triazine | | | | | | |
| Diethylamino hydroxybenzoyl | 1.0 | 1.0 | 1.0 | 1.0 | 1.0 | 1.5 |
| hexyl benzoate | | | | | | |
| Butyl octyl salicylate | 12.0 | 12.0 | 12.0 | 12.0 | 12.0 | 12.0 |
| Bisabolol | 1.0 | 1.0 | 1.0 | 1.0 | 1.0 | 1.0 |
| Ethylhexylglycerin | 1.0 | 1.0 | 1.0 | 1.0 | 1.0 | 1.0 |
| Tocopheryl acetate | 1.0 | 1.0 | 1.0 | 1.0 | 1.0 | 1.0 |

| | |
|---|---|
| Total | 100 |

### COMPARATIVE EXAMPLES

### Comparative Examples 1 to 4

As in Example 1, respective cosmetic compositions were prepared using components in the amounts shown in Table 2 below.

TABLE 2

| | Comparative Example (wt %) | | | |
|---|---|---|---|---|
| | 1 | 2 | 3 | 4 |
| Elastomer (B) | 55.0 | 55.0 | 55.0 | 55.0 |
| Ethylhexyl methoxycinnamate | 5.0 | 5.0 | 5.0 | 5.0 |
| Ethylhexyl salicylate | 5.0 | 5.0 | 5.0 | 5.0 |
| Homosalate | 7.0 | 7.0 | 7.0 | 7.0 |
| Polysilicone-15 | 4.0 | 4.0 | 4.0 | 4.0 |
| Isoamyl p-methoxycinnamate | 3.0 | 3.0 | 3.0 | 3.0 |
| Ethylhexyl triazone | 1.5 | 1.5 | 1.5 | 1.5 |
| Butyl methoxydibenzoylmethane | 1.0 | 1.0 | 1.0 | 1.0 |
| Bis-ethylhexyloxyphenol methoxyphenyl | 2.5 | 2.5 | 2.5 | 2.5 |
| triazine | | | | |
| Diethylamino hydroxybenzoyl hexyl | 1.0 | 1.0 | 1.0 | 1.0 |
| benzoate | | | | |
| Octocrylene | 4.0 | 1.0 | — | 1.0 |
| Titanium dioxide | — | — | 2.0 | 1.0 |
| Butyl octyl salicylate | 10.0 | 12.0 | 11.0 | 11.0 |
| Bisabolol | 0.5 | 0.5 | 0.5 | 0.5 |
| Ethylhexylglycerin | — | 1.0 | 1.0 | 1.0 |
| Tocopheryl acetate | 0.5 | 0.5 | 0.5 | 0.5 |

| | |
|---|---|
| Total | 100 |

### Comparative Examples 5 to 8

As in Example 1, respective cosmetic compositions were prepared using components in the amounts shown in Table 3 below.

TABLE 3

| | Comparative Example (wt %) | | | |
|---|---|---|---|---|
| | 5 | 6 | 7 | 8 |
| Elastomer (B) | 55.0 | 55.0 | 55.0 | 55.0 |
| Ethylhexyl methoxycinnamate | 6.0 | 6.5 | 7.0 | 7.0 |
| Ethylhexyl salicylate | 5.0 | 5.0 | 5.0 | 5.0 |
| Homosalate | 6.0 | 7.0 | 7.0 | 7.0 |
| Polysilicone-15 | — | 2.0 | 2.0 | 2.5 |
| Isoamyl p-methoxycinnamate | 2.0 | 2.0 | 5.0 | 5.0 |
| Ethylhexyl triazone | 2.0 | 2.0 | — | 1.0 |
| Butyl methoxydibenzoylmethane | 3.0 | 2.5 | 1.5 | — |
| Bis-ethylhexyloxyphenol methoxyphenyl | 3.0 | — | 1.5 | 1.5 |
| triazine | | | | |
| Diethylamino hydroxybenzoyl hexyl | 3.0 | 3.0 | 1.0 | 1.0 |
| benzoate | | | | |
| Butyl octyl salicylate | 12.0 | 12.0 | 12.0 | 12.0 |
| Bisabolol | 1.0 | 1.0 | 1.0 | 1.0 |
| Ethylhexylglycerin | 1.0 | 1.0 | 1.0 | 1.0 |
| Tocopheryl acetate | 1.0 | 1.0 | 1.0 | 1.0 |

| | |
|---|---|
| Total | 100 |

### Comparative Examples 9 to 14

As in Example 1, respective cosmetic compositions were prepared using components in the amounts shown in Table 4 below.

TABLE 4

| | Comparative Example (wt %) | | | | | |
|---|---|---|---|---|---|---|
| | 9 | 10 | 11 | 12 | 13 | 14 |
| Elastomer (B) | 30.0 | 75.0 | 60.0 | 40.0 | 55.0 | 40.0 |
| Ethylhexyl methoxycinnamate | 5.0 | 3.0 | 0.5 | 4.0 | 5.0 | 3.0 |
| Ethylhexyl salicylate | 5.0 | 3.0 | — | 5.5 | 5.0 | 5.0 |
| Homosalate | 7.0 | 1.0 | 0.1 | 7.0 | 7.0 | 5.0 |
| Polysilicone-15 | 4.0 | 1.0 | 0.5 | 4.0 | 4.0 | 4.0 |
| Isoamyl p-methoxycinnamate | 3.0 | 1.5 | 0.3 | 1.5 | 3.0 | 2.0 |
| Ethylhexyl triazone | 1.5 | 0.5 | 2.0 | 11.0 | 1.5 | 1.5 |
| Butyl | 1.0 | 1.0 | — | 5.0 | 1.0 | 1.0 |
| methoxydibenzoylmethane | | | | | | |
| Bis-ethylhexyloxyphenol | 2.5 | 0.5 | 0.1 | 11.0 | 2.5 | 2.5 |
| methoxyphenyl triazine | | | | | | |
| Diethylamino hydroxybenzoyl | 1.0 | 1.0 | — | 1.0 | 1.0 | 1.0 |
| hexyl benzoate | | | | | | |
| Butyl octyl salicylate | 25.0 | 12.5 | 25.0 | 10.0 | — | 35.0 |
| Bisabolol | 5.0 | — | 4.0 | — | 5.0 | — |
| Ethylhexylglycerin | 5.0 | — | 5.0 | — | 5.0 | — |
| Tocopheryl acetate | 5.0 | — | 2.5 | — | 5.0 | — |

| | |
|---|---|
| Total | 100 |

### Comparative Examples 15 to 18

Respective cosmetic compositions were prepared in the same manner as in Example 1, with the exception that elastomers shown in Table 5 below were used.

TABLE 5

| | Elastomer |
|---|---|
| Comparative Example 15 | Cetearyl dimethicone crosspolymer |
| Comparative Example 16 | Dimethicone/vinyl dimethicone crosspolymer |
| Comparative Example 17 | Dimethicone/PEG-10/15 crosspolymer |
| Comparative Example 18 | PEG/PPG-20/15 dimethicone |

### TEST EXAMPLES

### Test Example 1: Evaluation of Transparency

(1) Visual Evaluation of Transparency A

The transparency of the cosmetic compositions prepared in Examples and Comparative Examples was evaluated with the naked eye (50 adults with visual acuity ranging from 1.0 to 1.2). The results of average values thereof are shown in Table 6 below.

In the following Table 6, a transparency score closer to 10 means that the transparency is excellent.

TABLE 6

| | Transparency |
|---|---|
| Example 1 | 9.8 |
| Example 2 | 9.0 |
| Example 3 | 9.3 |
| Example 4 | 9.2 |
| Example 5 | 9.4 |
| Example 6 | 9.4 |
| Comparative Example 1 | 0 |
| Comparative Example 2 | 0 |
| Comparative Example 3 | 0 |
| Comparative Example 4 | 0 |
| Comparative Example 5 | — |
| Comparative Example 6 | — |
| Comparative Example 7 | 2.5 |
| Comparative Example 8 | 2.2 |
| Comparative Example 9 | 3.5 |
| Comparative Example 10 | — |
| Comparative Example 11 | 6.5 |
| Comparative Example 12 | — |
| Comparative Example 13 | — |
| Comparative Example 14 | 3.0 |
| Comparative Example 15 | 0.1 |
| Comparative Example 16 | 0.1 |
| Comparative Example 17 | 0.3 |
| Comparative Example 18 | 0.5 |

As is apparent from Table 6, the transparency of Examples 1 to 6 was much higher than that of Comparative Examples. In particular, based on the results of comparing Example 1 and Comparative Examples 1 to 4, when octocrylene and titanium dioxide were used, the transparency was significantly reduced.

Meanwhile, in Comparative Examples 5, 6, 10, 12 and 13 of Evaluation (1) above, transparency testing was impossible because the organic ultraviolet-screening agent composition was precipitated in the form of granules.

(2) Visual Evaluation of Transparency B (Image)

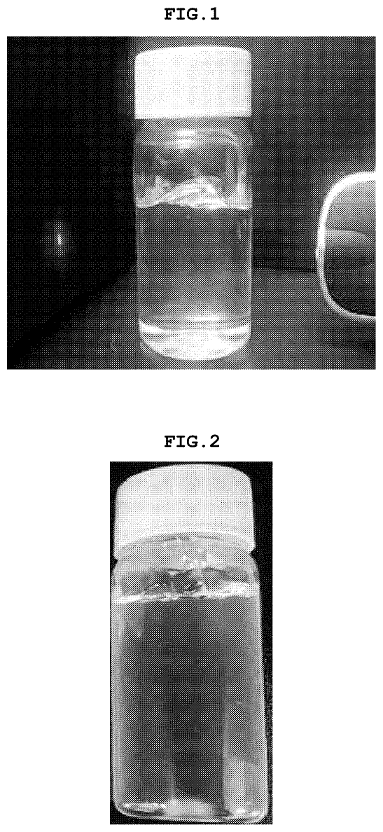

The images of the cosmetic compositions prepared in Examples and Comparative Examples are shown in FIGS. 1 to 17.

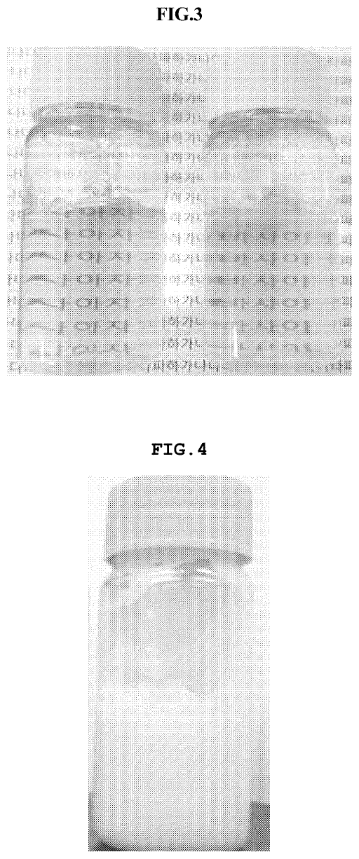

FIG. 1 shows Example 1, FIG. 2 shows Example 2, and FIG. 3 shows Examples 3 and 4, from which it can be confirmed that the cosmetic compositions of Examples are very transparent. In particular, Example 1 is very transparent to the extent that a red laser applied thereto passes through the cosmetic composition and shines on the wall, and Examples 3 and 4 are transparent such that the text behind the cosmetic composition can be clearly seen with the naked eye, indicating that the formation of a cosmetic composition in a very transparent formulation according to the present invention is possible.

FIG. 4 shows Comparative Example 1, which is dark yellowish and very opaque, and FIG. 5 shows Comparative Example 2, in which the transparency of the cosmetic composition is decreased and a yellowish color is apparent even upon the addition of a small amount of octocrylene.

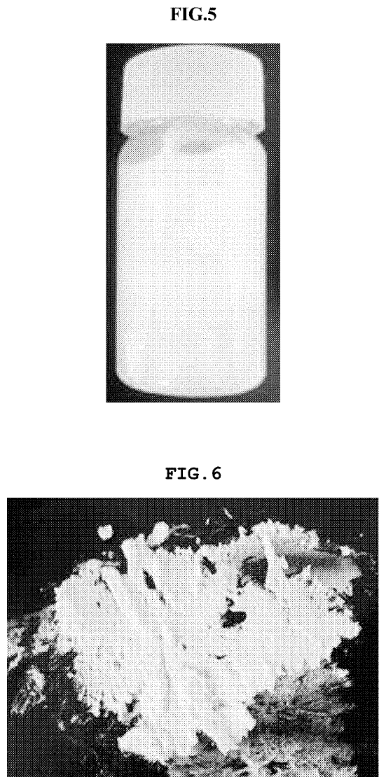

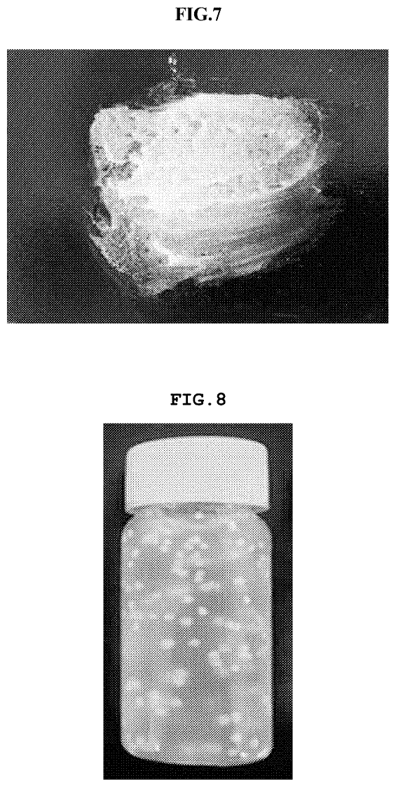

FIG. 6 shows Comparative Example 3, in which the formulation became opaque, stiff and brittle due to the use of titanium dioxide, and FIG. 7 shows Comparative Example 4, in which the formulation became opaque and stiff despite the use of octocrylene and titanium dioxide in smaller amounts compared to Comparative Examples 1 and 3.

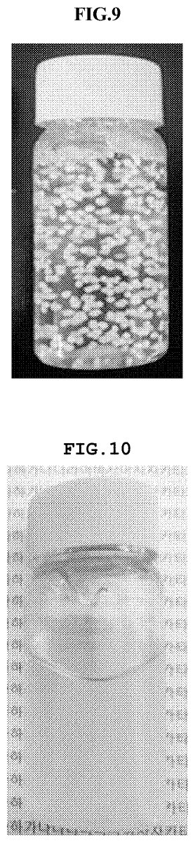

FIG. 8 shows Comparative Example 5, and FIG. 9 shows Comparative Example 6, in which granules were precipitated, both in Comparative Example 5, at a mixing weight ratio of 1.7:1 of the liquid organic ultraviolet-screening agent and the solid organic ultraviolet-screening agent in the organic ultraviolet-screening agent composition, and in Comparative Example 6, at a mixing weight ratio of 3:1 thereof.

FIG. 10 shows Comparative Example 7, in which the mixing weight ratio of the liquid organic ultraviolet-screening agent and the solid organic ultraviolet-screening agent in the organic ultraviolet-screening agent composition was 6.5:1, resulting in insufficient viscosity and an opaque cosmetic.

As shown in FIGS. 8 to 10, when the mixing weight ratio of the liquid organic ultraviolet-screening agent and the solid organic ultraviolet-screening agent in the organic ultraviolet-screening agent composition falls out of the range of 3.5-5:1, it can be confirmed that the transparency of the cosmetics is lowered, and the organic ultraviolet-screening agent components are not only precipitated in the form of granules but also the formulation is unstable.

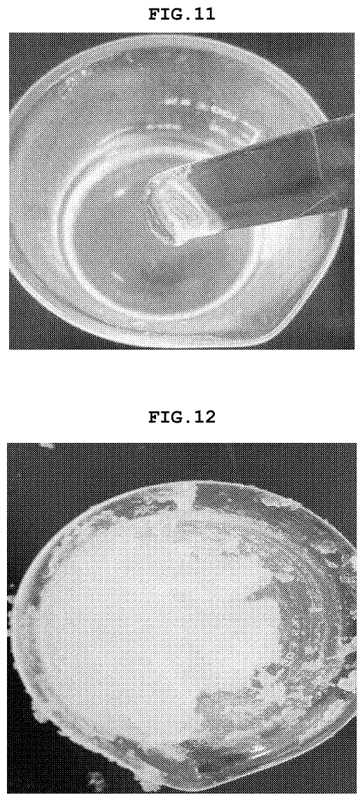

FIG. 11 shows Comparative Example 9. In the case of a small amount of elastomer, it can be confirmed that individual components are not sufficiently compatible (mixed), thus obtaining insufficient viscosity and sinking the elastomer to the bottom due to layer separation.

FIG. 12 shows Comparative Example 10. When the amount of the elastomer is excessively large and the amount of oil is relatively small, it can be confirmed that the organic ultraviolet-screening agent composition is precipitated in the form of fine granules, thereby decreasing transparency, and also that the flexibility of the formulation is lowered and thus the resulting cosmetic may become brittle because the amount of the oil is relatively lower than that of the elastomer.

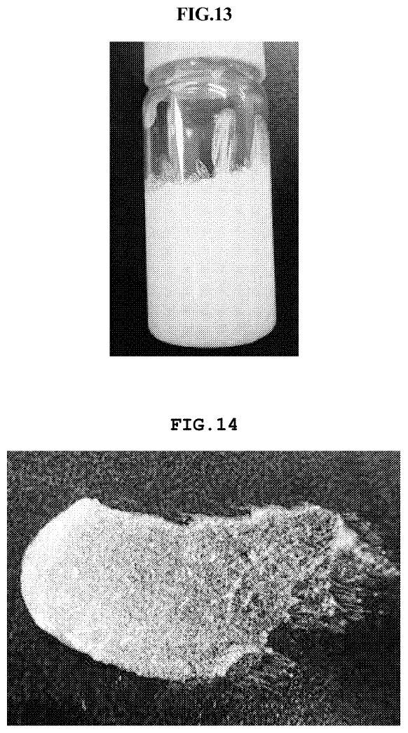

FIGS. 13 and 14 show Comparative Example 12. When an excess of the organic ultraviolet-screening agent composition is added, it can be confirmed that the organic ultraviolet-screening agent is precipitated in the form of granules and that the resulting cosmetic becomes opaque overall.

FIG. 15 shows Comparative Example 13. In the case in which no oil is added, it can be confirmed that fine granules are formed and the resulting cosmetic composition becomes opaque overall.

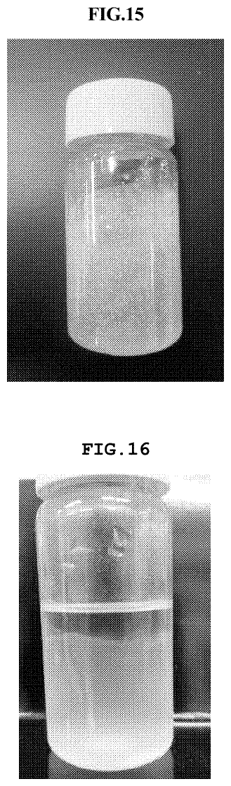

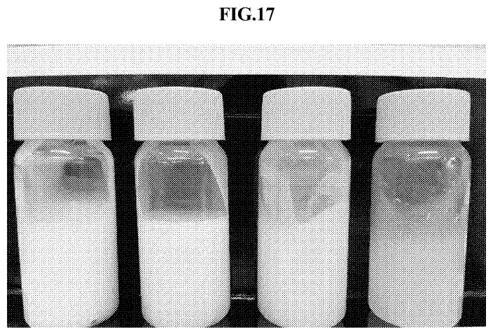

FIG. 16 shows Comparative Example 14. In the case in which an excess of oil is added, it can be confirmed that individual components are not sufficiently compatible (mixed) and thus layer separation occurs.

FIG. 17 shows Comparative Examples 15 to 18. In the case in which the dimethicone/bis-isobutyl PPG-20 crosspolymer and/or the PEG-12 dimethicone/PPG-20 crosspolymer were not used as the elastomer, even when the elastomer, the oil, and the organic ultraviolet-screening agent composition were mixed at an appropriate ratio, the resulting cosmetic composition became very opaque. FIG. 17 sequentially illustrates Comparative Example 15 (left), Comparative Example 16, Comparative Example 17, and Comparative Example 18 (right).

### Test Example 2: Ultraviolet (UV)-Screening Effect

The UV-screening effects of the cosmetic compositions prepared in Examples and Comparative Examples were measured using a UV2000S. The results thereof are shown in Table 7 below. In the following Table 7, the higher the UV-screening index and the critical wavelength, the better the UV-screening effect.

TABLE 7

| | UV-screening index | Critical wavelength |
|---|---|---|
| Example 1 | 72 | 377 |
| Example 2 | 66 | 376 |
| Example 3 | 68 | 377 |
| Example 4 | 68 | 376 |
| Example 5 | 70 | 376 |
| Example 6 | 70 | 376 |
| Comparative Example 1 | 75 | 377 |
| Comparative Example 2 | 68 | 376 |
| Comparative Example 3 | — | — |
| Comparative Example 4 | — | — |
| Comparative Example 5 | 30 | 350 |
| Comparative Example 6 | 35 | 353 |
| Comparative Example 7 | 21 | 346 |
| Comparative Example 8 | 16 | 345 |
| Comparative Example 9 | 10 | 333 |
| Comparative Example 10 | 25 | 343 |
| Comparative Example 11 | 12 | 310 |
| Comparative Example 12 | 30 | 342 |
| Comparative Example 13 | 37 | 345 |
| Comparative Example 14 | 26 | 340 |
| Comparative Example 15 | 43 | 361 |
| Comparative Example 16 | 46 | 360 |
| Comparative Example 17 | 50 | 365 |
| Comparative Example 18 | 43 | 360 |

As is apparent from Table 7, the transparent cosmetic compositions of Examples of the present invention can exhibit almost the same ultraviolet-screening effect as that of Comparative Examples 1 and 2 using octocrylene. According to the present invention, a cosmetic composition having a high ultraviolet-screening effect and taking the form of a transparent formulation can be manufactured.

Comparative Examples 3 and 4 were difficult to evaluate because the formulations were stiff and brittle due to the use of titanium dioxide.

### Test Example 3: Evaluation of Usability

The cosmetic compositions of Examples and Comparative Examples were supplied to individual groups (consisting of 10 females in their twenties and thirties) and used at the last stage of skin care for a total of 7 days to thereby evaluate the coverage of skin pores and irregularities, adhesion and white cast.

In the evaluation of coverage and adhesion, a score of 5 indicates excellent, a score of 4 indicates good, a score of 3 indicates fair, a score of 2 indicates poor, and a score of 1 indicates very poor. Also, in the evaluation of white cast, the case where a white cast occurred is evaluated as Ξ, the case where a white cast was insignificant or partially occurred is evaluated as Δ, and the case where no white cast occurred is evaluated as Y.

The scores were recorded daily for a total of 7 days, and the average score of the 7 days is shown in Table 8 below.

TABLE 8

| | Coverage | Adhesion | White cast |
|---|---|---|---|
| Example 1 | 4.50 | 4.18 | X |
| Example 2 | 4.18 | 4.01 | X |
| Example 3 | 4.33 | 4.03 | X |
| Example 4 | 4.31 | 4.02 | X |
| Example 5 | 4.47 | 4.15 | X |
| Example 6 | 4.45 | 4.14 | X |
| Comparative Example 1 | 1.85 | 1.14 | ◯ |
| Comparative Example 2 | 2.44 | 2.56 | ◯ |
| Comparative Example 3 | — | — | — |
| Comparative Example 4 | — | — | — |
| Comparative Example 5 | — | — | — |
| Comparative Example 6 | — | — | — |
| Comparative Example 7 | 1.20 | 1.11 | X |
| Comparative Example 8 | 0.55 | 0.22 | X |
| Comparative Example 9 | — | — | — |
| Comparative Example 10 | — | — | — |
| Comparative Example 11 | 3.41 | 3.20 | ◯ |
| Comparative Example 12 | — | — | — |
| Comparative Example 13 | — | — | — |
| Comparative Example 14 | — | — | — |
| Comparative Example 15 | 2.15 | 3.01 | ◯ |
| Comparative Example 16 | 2.46 | 2.99 | ◯ |
| Comparative Example 17 | 2.19 | 3.42 | ◯ |
| Comparative Example 18 | 2.11 | 3.05 | ◯ |

As is apparent from Table 8, in Examples of the present invention, coverage and adhesion were superior and there was almost no white cast, but in Comparative Examples, coverage and adhesion were very low and a white cast occurred. In particular, as shown in Table 8, in Comparative Examples 3, 4, 5, 6, 9, 10, 12, 13, and 14, it was difficult to evaluate the use sensation, adhesion and white cast due to the precipitation of granules or instability of the formulation.

Based on the results of comparing Example 1 and Comparative Examples 15 to 18, in the case in which the dimethicone/bis-isobutyl PPG-20 crosspolymer and/or the PEG-dimethicone/PPG-20 crosspolymer were not used as the elastomer, even when the elastomer, the oil, and the organic ultraviolet-screening agent composition were mixed at an appropriate ratio, the adhesion and use sensation of the cosmetic composition deteriorated and a white cast occurred.

As shown in Test Examples 1 to 3, it can be concluded that the transparent cosmetic composition of the present invention exhibits a high ultraviolet-screening effect, a transparent formulation and no white cast, and can also function as a makeup primer that conceals skin pores and irregularities to thus make the skin texture smooth.

It will be apparent to those skilled in the art that various modifications and variations can be made in the present invention without departing from the spirit or scope of the present invention. Therefore, it should be understood that the above-described embodiments are illustrative in all aspects and not restrictive. For example, each component described as a single entity may be embodied in a distributed state, and components described as being distributed may be embodied in a combined form.

The scope of the present invention is defined by the appended claims rather than the detailed description, and all changes or modifications derived from the meaning and scope of the claims and their equivalents are to be construed as being included within the scope of the present invention.

1. A transparent cosmetic composition having an ultraviolet-screening function, comprising:
an elastomer, an oil, and an organic ultraviolet-screening agent composition,
wherein the organic ultraviolet-screening agent composition comprises:
at least one liquid organic ultraviolet-screening agent selected from the group consisting of ethylhexyl methoxycinnamate, ethylhexyl salicylate, homosalate, polysilicone-15, and isoamyl p-methoxycinnamate; and
at least one solid organic ultraviolet-screening agent selected from the group consisting of ethylhexyl triazone, butyl methoxydibenzoylmethane, bis-ethylhexyloxyphenol methoxyphenyl triazine and diethylamino hydroxybenzoyl hexyl benzoate,
wherein a mixing weight ratio of the at least one liquid organic ultraviolet-screening agent to the at least one solid organic ultraviolet-screening agent is a range of 3.5 to 5:1.

2. The transparent cosmetic composition of claim 1, wherein the transparent cosmetic composition comprises, based on a total weight of the transparent cosmetic composition, 35 to 70 wt % of the elastomer, 1 to 30 wt % of the oil and 5 to 40 wt % of the organic ultraviolet-screening agent composition.

3. The transparent cosmetic composition of claim 1, wherein the elastomer includes at least one selected from the group consisting of (A) a dimethicone/bis-isobutyl PPG-20 crosspolymer and (B) a PEG-12 dimethicone/PPG-20 crosspolymer.

4. (canceled)

5. The transparent cosmetic composition of claim 1, wherein the organic ultraviolet-screening agent composition includes, based on a total weight of the transparent cosmetic composition, 3 to 20 wt % of ethylhexyl methoxycinnamate, 3 to 20 wt % of ethylhexyl salicylate, 0.1 to 20 wt % of homosalate, 0.1 to 20 wt % of polysilicone-15, 0.1 to 20 wt % of isoamyl p-methoxycinnamate, 0.1 to 10 wt % of ethylhexyl triazone, 0.1 to 10 wt % of butyl methoxydibenzoylmethane, 0.1 to 10 wt % of bis-ethylhexyloxyphenol methoxyphenyl triazine, and 0.1 to 10 wt % of diethylamino hydroxybenzoyl hexyl benzoate.

6. The transparent cosmetic composition of claim 1, wherein the oil includes at least one selected from the group consisting of butyloctyl salicylate, dibutyl adipate, butylene glycol dicaprylate/dicaprate, caprylyl methicone, isododecane, isohexadecane, C15-19 alkane and dicaprylyl carbonate.

7. The transparent cosmetic composition of claim 1, further comprising at least one additive selected from the group consisting of a preservative, a whitening agent, an antioxidant, a skin-conditioning agent, and a surfactant.
